(19) United States
(12) Patent Application Publication
(10) Pub. No.: US 2023/0225822 A1
(43) Pub. Date: Jul. 20, 2023

(54) Optical Tracking of Objects in Arthroscopic Surgery

(71) Applicants: SMITH & NEPHEW, INC., Memphis, TN (US); SMITH & NEPHEW ORTHOPAEDICS AG, Zug (CH); SMITH & NEPHEW ASIA PACIFIC PTE. LIMITED, Singapore (SG)

(72) Inventors: Paul Alexander TORRIE, Marbelhead, MA (US); Constantinos NIKOU, Monroevill, PA (US); Nathan Anil NETRAVALI, Littleton, MA (US); Nicholas Ryan LABRIOLA, Andover, MA (US)

(73) Assignees: SMITH & NEPHEW, INC., Memphis, TN (US); SMITH & NEPHEW ORTHOPAEDICS AG, Zug (CH); SMITH & NEPHEW ASIA PACIFIC PTE. LIMITED, Singapore (SG)

(21) Appl. No.: 18/002,222

(22) PCT Filed: Jun. 30, 2021
(86) PCT No.: PCT/US21/39853

§ 371 Date: Dec. 16, 2022

Related U.S. Application Data
(60) Provisional application No. 63/119,133, filed on Nov. 30, 2020.
(60) Provisional application No. 63/047,317, filed on Jul. 2, 2020.

(51) Int. Cl. A61B 90/00; A61B 17/32

(52) U.S. Cl. CPC A61B 90/39; A61B 17/320016; A61B 2017/00477

(57) ABSTRACT

Optical tracking of objects in arthroscopic surgery. Examples comprise a resection instrument system including: a handpiece; a mechanical resection device comprising a stationary outer hub, an elongate outer tube coupled to and extending away from the stationary outer hub, and a cutter disposed at a distal end of the elongate shaft, the stationary outer hub coupled to the handpiece; a fiducial array; and a sleeve connector. The sleeve connector may include: a sleeve defining a distal end, a proximal end, and a through bore, the sleeve concentrically arranged with the elongate shaft; an array connector coupled to the proximal end of the sleeve, the array connector coupled to the fiducial array.

### CROSS-REFERENCE TO RELATED APPLICATIONS

This application claims the benefit of U.S. Provisional App. No. 63/047,317 filed Jul. 2, 2020 title “Tracked Cutter” and U.S. Provisional App. No. 63/119,133 filed Nov. 30, 2020 titled “Multiplanar Optical Tracking Arrays.” Both provisional applications are incorporated by reference herein as if reproduced in full below.

### BACKGROUND

Robotic surgery may use an optical tracking system to track the location of bones and instruments in the three-dimensional space of the surgical room. Optical tracking is a proven technology where the reflectors are rigidly connected to the working end of the tracked instrument. Similarly, reflectors are rigidly connected to bones to track location of the bones during the surgical procedure.

Related-art reflectors have a limited range of visibility with respect to the optical tracking system. That is, the related-art reflectors are only visible to the stereoscopic camera of the optical tracking system in a relatively narrow range of viewing angles. For surgeries where the range of the motion of the tracked instrument is relatively narrow (e.g., knee arthroplasty), the narrow range of viewing angles does not present a major issue. However, in other surgical cases, such as treating femoroacetabular impingement, the range of motion of the tracked instrument to remove bone from around the femoral neck, for example, makes tracking the instrument difficult.

### SUMMARY

One example is a resection instrument system comprising: a handpiece; a mechanical resection device comprising a stationary outer hub, an elongate outer tube coupled to and extending away from the stationary outer hub, and a cutter disposed at a distal end of the elongate outer tube, the stationary outer hub coupled to the handpiece; a fiducial array; and a sleeve connector. The sleeve connector may comprise: a sleeve defining a distal end, a proximal end, and a through bore, the sleeve concentrically arranged with the elongate outer tube; and an array connector coupled to the proximal end of the sleeve, the array connector coupled to the fiducial array.

In the example resection system, the array connector may further comprise: a first abutment surface configured to abut a first complementary surface of the fiducial array; a second abutment surface configured to abut a second complementary surface of the fiducial array; a third abutment surface configured to about a third complementary surface of the fiducial array; and a latch configured to hold the fiducial array in an abutting relationship with the first, second, and third abutment surfaces.

In the example resection system, the array connector may further comprise: a slot formed by the array connector; and a fiducial connector of the fiducial array disposed within the slot.

In the example resection system, the array connector may further comprise: a first arm; a second arm; and an intersection defined by the first and second arms, and wherein the fiducial connector abuts the intersection. A distance between the first arm and the second arm may increase with increasing distance away from the proximal end of the sleeve. The first arm and the second arm may be coplanar, and a plane defined by the first and second arms may be parallel to a longitudinal central axis of the through bore of the sleeve.

In the example resection system, the array connector may further comprise: a first notch, and a first portion of the fiducial array disposed with the first notch; a second notch, and a second portion of the fiducial array disposed with the second notch. The first portion of the fiducial array may elastically deform the first notch and the second portion of the fiducial array elastically deforms the second notch.

In the example resection system, sleeve connector may further comprise: a first latch disposed proximate to a receptacle on the proximal end of the sleeve, the first latch configured to hold the sleeve at a fixed axial position relative to the stationary outer hub when the sleeve is located concentrically around the elongate outer tube; and a second latch disposed proximate to the receptacle, the second latch configured to hold the sleeve at the fixed axial position relative to the stationary outer hub when the sleeve is concentrically disposed around the elongate outer tube. The first latch may further comprise a first latch arm having a first length measured from a hinge of the first latch. The second latch may further comprise a second latch arm having a second length measured from a hinge of the second latch, the second length longer than the first length.

In the example resection system, the sleeve connector may further comprise a counter bore on proximal end of the sleeve, the counter bore defining an inside diameter greater than an outside diameter of the stationary outer hub.

The example resection system may further comprise: the sleeve defines a radius of curvature with a sleeve center of curvature outside the sleeve; the elongate outer tube a radius of curvature with a tube center curvature outside the elongate outer tube; and the sleeve center of curvature and the tube center of curvature are co-located.

In the example resection system, the fiducial array may further comprise: a first set of reflector receptacles, the first set of reflector receptacles define and reside in a first plane; a first set of fiducials disposed one each in association with each of the first set of reflector receptacles; a second set of reflector receptacles, the second set of reflector receptacles define and reside in a second plane; a second set of fiducials disposed one each in association with each of the second set of reflector receptacles; a third set of reflector receptacles, the third set of reflector receptacles define and reside in a third plane; a third set of fiducials disposed one each in association with each of the third set of reflector receptacles; and an array structure that holds the first set of reflector receptacles, the second set of reflector receptacles, and the third set of reflector receptacles in a fixed orientation. The example resection system may further comprise at least three reflector receptacles in each of the first, second, and third sets of reflector receptacles. The second plane and the third plane may be parallel, and the first plane may be perpendicular to both the second plane and the third plane. In some cases, at least one reflector receptacle of the second set of reflector receptacles is shared with the third set of reflector receptacles. In the example resection system, the fiducial array may comprise: the first set of reflector receptacles defines a first pattern; the second set of reflector receptacles defines a second pattern different than the first pattern; and the third set of reflector receptacles defines a third pattern different than the first and second patterns. In the example resection system, the fiducial array may further comprise the first plane intersects the second plane, the second plane intersects the third plane, and the third plane intersects the first plane. The third plane may intersect the first plane between reflector receptacles of the first set of reflector receptacles, and the third plane may intersect the second plane between reflector receptacles of the second set of reflector receptacles. The third plane may intersect the first plane outside a boundary defined by first set of reflector receptacles, and the third plane may intersect the second plane outside a boundary defined by the second set of reflector receptacles.

In the example resection system, the fiducial array may further comprise a fiducial reflector, and the reflector may comprise: a body defining a front surface, a back surface, and a central axis; a front reflector disposed on the front surface; and a first annular latch partially circumscribing the body, the first annular latch comprising a hinge proximate to back surface, a cantilever projecting toward the front surface, and a catch surface parallel to the front surface. The example resection system may further comprise a second annular latch partially circumscribing the body, the second annular latch comprising a hinge proximate to front back surface, a cantilever projecting toward the back surface, and a catch surface parallel to the back surface. In some cases, the front surface and the back surface are parallel. The fiducial reflector may further comprise a back reflector disposed on the back surface. The first annular latch may further comprise a first latch member and a second latch member, each of the first and second latch members comprising a hinge proximate to back surface, a cantilever projecting toward the front surface, and a catch surface parallel to the front surface. The fiducial reflector may further comprise a second annular latch, and the second annular latch may further comprise a third latch member and a fourth latch member, each of the third and fourth latch members comprising a hinge proximate to the front back surface, a cantilever projecting toward the back surface, and a catch surface parallel to the back surface. The second latch member may be disposed between the first and second latch members. The example fiducial connector may further comprise a latch indicator disposed within an annular channel defined between the first annular latch and the body, the latch indicator visible when the first annular latch is in a non-deflected orientation

Another example is a receptacle array comprising: a first set of reflector receptacles, the first set of reflector receptacles define and reside in a first plane; a second set of reflector receptacles, the second set of reflector receptacles define and reside in a second plane; a third set of reflector receptacles, the third set of reflector receptacles define and reside in a third plane; and an array structure that holds the first set of reflector receptacles, the second set of reflector receptacles, and the third set of reflector receptacles in a fixed orientation.

The example receptacle array may further comprise at least three reflector receptacles in each of the first, second, and third sets of reflector receptacles.

The example receptacle array may further comprise: the second plane and the third plane are parallel; and the first plane is perpendicular to both the second plane and the third plane.

The example receptacle array may further comprise at least one reflector receptacle of the second set of reflector receptacles is shared with the third set of reflector receptacles.

The example receptacle array may further comprise: the first set of reflector receptacles define a first pattern; the second set of reflector receptacles define a second pattern different than the first pattern; and the third set of reflector receptacles define a third pattern different than the first and second patterns.

In the example receptacle array, the first plane may intersect the second plane, the second plane may intersects the third plane, and the third plane may intersects the first plane. The third plane may intersects the first plane between reflector receptacles of the first set of reflector receptacles, and the third plane may intersects the second plane between reflector receptacles of the second set of reflector receptacles. The third plane may intersect the first plane outside a boundary defined by the first set of reflector receptacles, and the third plane may intersect the second plane outside a boundary defined by the second set of reflector receptacles.

Yet another example is a fiducial reflector, comprising: a body defining a front surface, a back surface, and a central axis; a front reflector disposed on the front surface; a first annular latch partially circumscribing the body, the first annular latch comprising a hinge proximate to back surface, a cantilever projecting toward the front surface, and a catch surface parallel to the front surface; and a second annular latch partially circumscribing the body, the second annular latch comprising a hinge proximate to front back surface, a cantilever projecting toward the back surface, and a catch surface parallel to the back surface.

In the example fiducial reflector, the front surface and the back surface may be parallel.

The example fiducial reflector may further comprise a back reflector disposed on the back surface.

In the example fiducial reflector the first annular latch may further comprise a first latch member and a second latch member, each of the first and second latch members comprising a hinge proximate to back surface, a cantilever projecting toward the front surface, and a catch surface parallel to the front surface. And the example fiducial reflector may further comprise: a second annular latch may further comprise a third latch member and a fourth latch member, each of the third and fourth latch members comprising a hinge proximate to the front back surface, a cantilever projecting toward the back surface, and a catch surface parallel to the back surface; and the second latch member disposed between the first and second latch members.

The example fiducial reflector may further comprise a latch indicator disposed within an annular channel defined between the first annular latch and the body, the latch indicator visible when the first annular latch is in a non-deflected orientation

Yet another example is a sleeve connector for a fiducial array, the sleeve connector comprising: a sleeve defining a distal end, a proximal end, and a through bore; an array connector coupled to the proximal end of the sleeve, and the array connector projecting away from the distal end of the sleeve. The array connector may comprise: a first abutment surface configured to abut a first complementary surface of a fiducial array; a second abutment surface configured to abut a second complementary surface of the fiducial array; and a third abutment surface configured to about a third complementary surface of the fiducial array; and a latch configured to hold the fiducial array in an abutting relationship with the first, second, and third abutment surfaces.

The example is a sleeve connector may further comprise a first notch disposed proximate to the first abutment surface. The example is a sleeve connector may further comprise a second notch disposed proximate to the second abutment surface.

The example is a sleeve connector may further comprise a first latch disposed at the proximal end of the sleeve, the first latch configured to hold the sleeve at a fixed axial position relative to a stationary outer hub of a mechanical resection device when the sleeve is concentrically disposed over an elongate outer tube of the mechanical resection device. The first latch may further comprise a first latch arm having a first length measured from a hinge of the first latch. The example is a sleeve connector may further comprise a second latch disposed between the proximal end of the sleeve and the second arm, the second latch configured to hold the sleeve at the fixed axial position relative to the stationary outer hub when the sleeve is concentrically disposed over the elongate outer tube of the mechanical resection device. The second latch may further comprise a second latch arm having a second length measured from a hinge of the second latch, the second length longer than a first length of a first latch arm.

The example is a sleeve connector may further comprise a counter bore on proximal end of the sleeve, the counter bore defining an inside diameter greater than an outside diameter of the sleeve.

The example is a sleeve connector may further comprise a spring in operational relationship to the through bore, the spring configured to at least one selected from a group comprising: increase a holding force of the sleeve to an elongate outer tube of a mechanical resection device; and provide a net force tending to center the sleeve concentrically over the elongate outer tube of the mechanical resection device.

In the example is a sleeve connector the through bore of the sleeve may define a radius of curvature with a center outside the sleeve.

### BRIEF DESCRIPTION OF THE DRAWINGS

For a detailed description of example embodiments, reference will now be made to the accompanying drawings in which:

FIG. 1 shows a robotic surgical system in accordance with at least some embodiments;

FIG. 2 shows an exploded perspective view of a resection instrument system in accordance with at least some embodiments;

FIG. 3 shows an exploded perspective view of the sleeve connector and the instrument fiducial array in accordance with at least some embodiments;

FIG. 4 shows a side elevation view of the resection instrument system in assembled form, and in accordance with at least some embodiments;

FIG. 5 shows a partial cross-sectional view of the array connector mated with the fiducial connector of the instrument fiducial array, in accordance with at least some embodiments;

FIG. 6 shows a partial front perspective view of the connection between the array connector and the fiducial connector in accordance with at least some embodiments;

FIG. 7 shows a partial back perspective view of the connection between the array connector and the fiducial connector, in accordance with at least some embodiments;

FIG. 8 shows a partial cross-sectional view of a resection instrument system in accordance with at least some embodiments;

FIG. 9 shows a sleeve connector in accordance with at least some embodiments;

FIG. 10 shows a perspective view of the instrument fiducial array in accordance with at least some embodiments;

FIG. 11A shows a back perspective view of a fiducial array in accordance with at least some embodiments;

FIG. 11B shows a front elevation view of the fiducial array of FIG. 11A, and in accordance with at least some embodiments;

FIG. 12 shows a perspective view of a fiducial in accordance with at least some embodiments;

FIG. 13 shows a cross-sectional view of a fiducial in accordance with at least some embodiments;

FIG. 14A shows a cross-sectional view of a fiducial and reflector receptacle in accordance with at least some embodiments;

FIG. 14B shows a cross-sectional view of a fiducial installed within a reflector receptacle in accordance with at least some embodiments; and

FIG. 15 shows a cross-sectional view of a fiducial with latch members deflected, in accordance with at least some embodiments.

### DEFINITIONS

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection or through an indirect connection via other devices and connections.

“Through bore” shall mean an aperture or passageway through an underlying device. However, the term “through bore” shall not be read to imply any method of creation. Thus, a through bore may be created in any suitable way, such as drilling, boring, laser drilling, or casting.

“Counter bore” shall mean an aperture or passageway into an underlying device. In cases in which the counter bore intersects another aperture (e.g., a through bore), the counter bore may thus define an internal shoulder. However, the term “counter bore” shall not be read to imply any method of creation. A counter bore may be created in any suitable way, such as drilling, boring, laser drilling, or casting.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Various examples are directed to optical tracking of objects in arthroscopic surgical procedures. Some examples are directed to sleeve connectors and associated fiducial arrays used to track location of mechanical resection devices where the mechanical resection devices are not specifically designed for use with optical tracking systems. Use of the sleeve connectors and associated fiducial arrays enables tracking location of a cutter of the mechanical resection devices where the tracking is not adversely affected by any movement in the connection between the mechanical resection device and the handpiece. Other examples are fiducial arrays having multiple sets of reflectors disposed respectively on various intersecting and/or non-intersecting planes to increase the visibility of the fiducial array to a stereoscopic camera through greater range of motion. The specification first turns to a description of an example system to orient the reader.

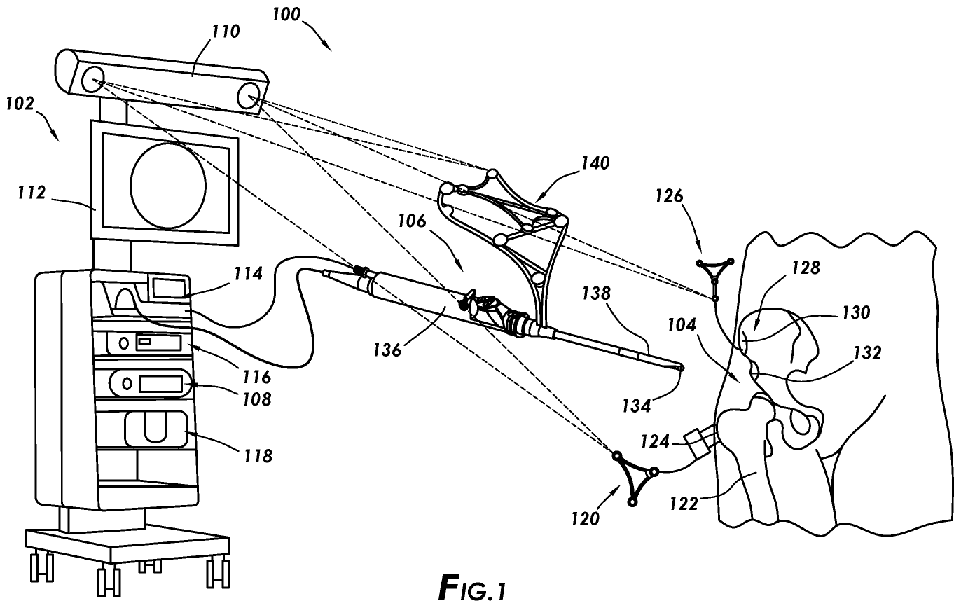

FIG. 1 shows a robotic surgical system in accordance with at least some embodiments. In particular, robotic surgical system 100 comprises a device cart 102, an example patient showing the patient's hip joint 104, and a resection instrument system 106. The device cart 102 may comprise a procedure controller 108, a stereoscopic camera 110 coupled to the procedure controller 108, a display device 112 coupled to the procedure controller 108, and a resection controller 114 communicatively coupled to the procedure controller 108. Other devices and controllers may be present as part of the device cart 102, such as an endoscopic light source and video controller 116 (hereafter just video controller 116). The endoscope associated with the video controller 116 is not shown in FIG. 1 so as not to further complicate the figure. Additional devices disposed on the device cart 102 may comprise a peristaltic pump system 118 which may be used to control inflow and outflow within the hip joint 104. Fluidic connections of the peristaltic pump systems 118 to the hip joint 104 are not shown so as not to further complicate the figure. The example device cart 102 shows only a single display device 112 used by the procedure controller 108; however, in practice a second display device may be present to show video images created by the endoscope and video controller 116. In yet still other cases, the display device 112 may be shared by the procedure controller 108 and the video controller 116 associated with the endoscope.

The stereoscopic camera 110 may take any suitable form. In some cases, the stereoscopic camera 110 is designed and constructed to receive light within the infrared (IR) band of frequencies, but in other cases the stereoscopic camera 110 may be operable with light in the visible range, or both. Regardless, in being stereoscopic, the stereoscopic camera 110 may be used by the procedure controller 108 to monitor location of various devices and structures in a three-dimensional coordinate space of the surgical room. That is, example systems either operate based on ambient light within the surgical room, or shine light toward the surgical procedure (e.g., IR frequencies). The light of interest is reflected by reflectors, fiducials, or fiducial arrays, and based on the reflected light the procedure controller 108 may determine the location of the fiducial arrays (and their attached devices/structures) within the surgical room. For example, prior to the example resection the surgeon may mechanically and rigidly couple a femur fiducial array 120 to the femur 122, such as by coupling the femur fiducial array 120 to the greater trochanter 124 of the femur 122. Once the femur fiducial array 120 is attached, and the femur 122 is correlated or registered within the system, the procedure controller 108 may monitor location of the femur fiducial array 120, and thus the femur 122, within the three-dimensional coordinate space of the surgical room.

As another example of monitoring location of various devices and structures, prior to resection the surgeon may mechanically and rigidly couple an acetabular fiducial array 126 to the acetabulum 128. The acetabular fiducial array 126 may be coupled at any suitable location, such as the superior iliac spine 130 or the inferior iliac spine 132, or both. Once the acetabular fiducial array 126 is coupled to the acetabulum 128, and the acetabulum 128 is correlated or registered within the system, the procedure controller 108 may monitor location of the acetabular fiducial array 126, and thus the acetabulum 128, within the three-dimensional coordinate space of the surgical room. While FIG. 1 shows a system in which the procedure controller 108 monitors location of the both the femur 122 and the acetabulum 128, in some case only one member of the hip joint 104 may be monitored, such as monitoring just the femur 122 when only a cam-deformity is to be resected as part of treating femoroacetabular impingement, or monitoring just the acetabulum 128 when only a pincer-deformity is to be resected as part of treating a femoroacetabular impingement.

Still referring to FIG. 1, the procedure controller 108 may further monitor, using the stereoscopic camera 110, location of the resection instrument system 106 operatively coupled to the resection controller 114. More particularly, in example systems the procedure controller 108 may monitor location of a cutter 134 on a distal end of the resection instrument system 106, such as to track the location and amount of bone removed during resection. The example resection instrument system 106 comprises a motor drive unit (MDU) or handpiece 136, a shaft or elongate outer tube 138 of a mechanical resection device (discussed more below), and the cutter 134 disposed at the distal end of the elongate outer tube 138. In one example, the cutter 134 is burr, but any suitable mechanical, radio-frequency resection, or laser resection device may be used. In order to track the distal end (e.g., the cutter 134) of the resection instrument system 106, an instrument fiducial array 140 is coupled to the elongate outer tube 138 and cutter 134, and the instrument fiducial array 140 is in optical view of the stereoscopic camera 110.

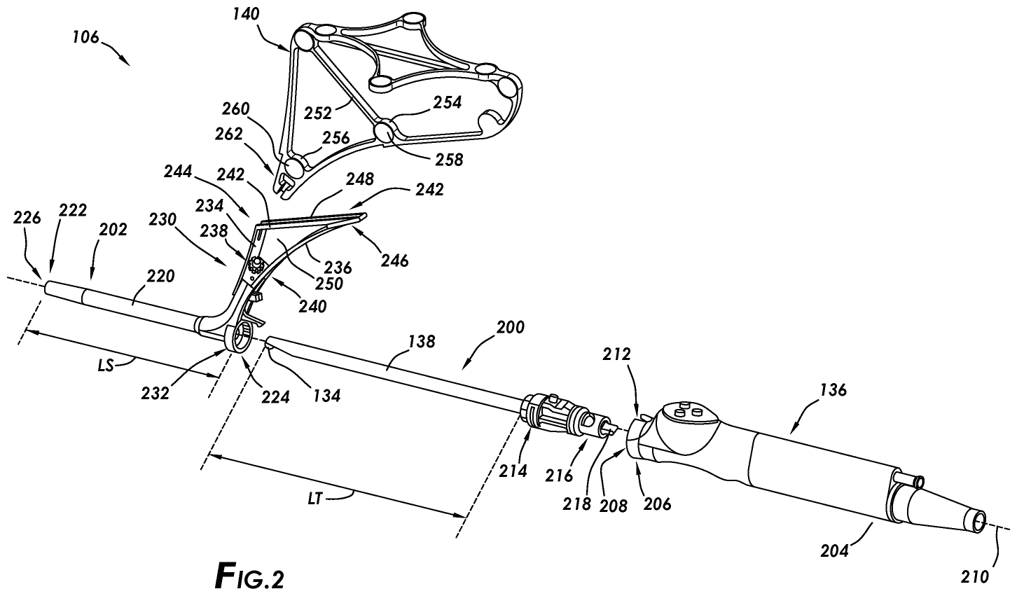

FIG. 2 shows an exploded perspective view of a resection instrument system in accordance with at least some embodiments. In particular, FIG. 2 shows the example resection instrument system 106 including the handpiece 136, a mechanical resection device 200 including the elongate outer tube 138, and the instrument fiducial array 140. Better shown in FIG. 2 is a sleeve connector 202 that couples the instrument fiducial array 140 to the mechanical resection device 200. The discussion starts with the handpiece 136 and works to the left. The example handpiece 136 comprises a proximal end 204 and a distal end 206. Defined on the distal end 206 is a socket or receptacle 208. Disposed within receptacle 208 is a rotational connector (not visible in FIG. 2) coupled to a drive shaft of a motor (not visible), such as an electric motor, disposed within the handpiece 136. The motor within the handpiece 136 and the rotational connector within the receptacle 208 are designed and constructed to provide rotational energy to the mechanical resection device 200. In the example system, the mechanical resection device 200 and the handpiece 136 are designed and constructed to enable the mechanical resection device 200 to couple to the handpiece 136 in one of two rotational orientations with respect to the longitudinal central axis 210. From the perspective of the example handpiece 136, the rotational orientations are defined by two slots through a side wall of the receptacle 208. In the view of FIG. 2, only one slot is visible, being slot 212 defined on the upper surface of the receptacle 208. A corresponding slot will be present through the sidewall of the receptacle 208 on the opposite side of the receptacle 208.

The example mechanical resection device 200 comprises a connector or stationary outer hub 214 rigidly coupled to the elongate outer tube 138. The stationary outer hub 214 defines an internal volume fluidly coupled an internal volume defined by an inside diameter of the elongate outer tube 138. Though not visible in FIG. 2, an internal tube or cylinder is telescoped within and concentrically arranged within the elongate outer tube 138, and the cylinder is mechanically coupled to the cutter 134 to provide rotational energy to the cutter 134. The cylinder is rigidly coupled to a rotating connector or inner hub 216 disposed at least partially within the stationary outer hub 214, and the inner hub 216 defines an example drive tang 218. The drive tang 218 is designed and constructed to couple to the rotational connector within the receptacle 208, and by way of the rotational connector and drive tang 218 the motor within the handpiece 136 provides rotational energy to the cutter 134. Thus, during assembly of the example resection instrument system 106 the inner hub 216 and stationary outer hub 214 are telescoped within receptacle 208 along the longitudinal central axis 210 to create the physical connection between the mechanical resection device 200 and the handpiece 136.

Still referring to FIG. 2, the example resection instrument system 106 further comprises the sleeve connector 202. The example sleeve connector 202 includes a sleeve 220 defining a distal end 222, a proximal end 224, and a through bore 226. The through bore resides along and/or defines the longitudinal central axis 210 of the sleeve 220. The example sleeve connector 202 further comprises an array connector 230 projecting out over the longitudinal central axis 210. When assembled, the array connector 230 extends over the handpiece 136. In the example system shown, the array connector 230 extends directly over the longitudinal central axis 210, and when assembled directly over the handpiece 136, but in other cases a plane defined by the array connector 230 may form a non-zero angle with the longitudinal central axis 210. As the name implies, the array connector 230 is designed and constructed to couple to the instrument fiducial array 140.

In accordance with example systems, the sleeve 220 of the sleeve connector 202 is a cylinder of metallic material (e.g., surgical grade stainless steel) suitable for sterilization by way of an autoclave. The sleeve 220 defines an outside surface, an inside surface, and a length LS. In example cases, the dimension of the inside surface of the sleeve 220 is designed and constructed to form a slip fit with the outside surface of the elongate outer tube 138. The length LS of the sleeve 220 is selected to be shorter than a length LT of the elongate outer tube 138. In one example case, the length LS is between and including one-half and three-quarters of the length LT of the elongate outer tube 138, but the length LS may be any length in which the cutter 134 is not obscured by the distal end 222 of the sleeve 220. The combination of the wall thickness of the sleeve 220 and the length LS of the sleeve 220 stiffen the mechanical resection device 200 to reduce the amount of deflection of the cutter 134 (e.g., relative to the longitudinal central axis 210) when bending loads are placed upon the elongate outer tube 138. Stated otherwise, the location of the cutter 134 in relation to the instrument fiducial array 140 (when the resection instrument system 106 is fully assembled) is assumed by the procedure controller 108 (FIG. 1) to be fixed based on a calibration and registration process. Thus, changes of the location of the cutter 134 relative to the instrument fiducial array 140 introduces error into the system, and the sleeve 220 stiffening the mechanical resection device 200 reduces system error.

In example cases the cross-sectional shape of the inside surface of the sleeve 220 is selected to match the cross-sectional shape of the outside surface of the elongate outer tube 138. In many cases, the outside surface of the elongate outer tube 138 is a right circular cylinder, and thus the outside surface of the elongate outer tube 138 defines a circular cross-section perpendicular to the longitudinal central axis 210. The sleeve 220 defines an inside diameter selected to form a slip fit with the elongate outer tube 138. During assembly, the sleeve 220 telescopes over the elongate outer tube 138 along the longitudinal central axis 210, and when fully seated against the stationary outer hub 214, the sleeve 220 is concentrically disposed around the elongate outer tube 138.

In the example case of FIG. 2, the sleeve 220 defines receptacle 232 on the proximal end 224 of the sleeve 220. The receptacle 232 has an outside diameter greater than an outside diameter of the stationary outer hub 214, and the receptacle 232 includes a counter bore defining an inside diameter greater than the outside diameter of the stationary outer hub 214. During assembly the receptacle 232 telescopes over at least a portion of the stationary outer hub 214. In some cases, the receptacle 232 and stationary outer hub 214 are keyed such that the receptacle telescopes over and is concentrically arranged with the distal end of the stationary outer hub 214 such that the window at the distal end of the elongate outer tube 138 has a fixed rotational relationship with the sleeve connector 202 and the instrument fiducial array 140. Stated differently, the receptacle 232 and stationary outer hub 214 may be keyed in such a way that the sleeve connector 202 couples to the mechanical resection device 200 in only two rotational orientations such that the exposed portion of the cutter 134 through a window of the elongate outer tube 138 is predetermined with respect to the location of the instrument fiducial array 140. The keyed connection may take any suitable form. For example, a distal end of the stationary outer hub 214 may define flat surface at a fixed position relative to the window at the distal end of the elongate outer tube 138 (e.g., aligned radially with the exposed portion of the cutter), and the receptacle 232 may have a corresponding flat surface on the inside surface of the receptacle. Stated more generically, the distal end of the stationary outer hub 214 may form a cross-sectional shape that telescopes into the receptacle in only two rotational orientations relative to the longitudinal central axis of the mechanical resection device, and the inside surface of the receptacle 232 may comprise a complementary cross-sectional shape.

Still referring to FIG. 2, and specifically now the array connector 230. The example array connector 230 comprises a first arm member 234 (hereafter just first arm 234) and a second arm member 236 (hereafter just second arm 236). The first arm 234 couples on a proximal end 238 to the proximal end 224 of the sleeve 220, and the first arm 234 extends away from the longitudinal central axis 210. The second arm 236 couples on a proximal end 240 to the proximal end 224 of the sleeve 220, and the second arm 236 extends away from the longitudinal central axis 210. The proximal ends 238 and 240 of the first and second arms 234 and 236, respectively, form an intersection (in combination with a stationary component of a latch, discussed more below).

At the opposite end of the array connector 230 from the intersection, a first support member 242 is coupled between a distal end 244 of the first arm 234 and a distal end 246 of the second arm 236. Similarly, a second support member 248 is coupled between the distal end 244 of the first arm 234 and the distal end 246 of the second arm 236. The first and second support members 242 and 248, along with the first and second arms 234 and 236, define a receptacle or slot 250 into which a portion of the instrument fiducial array 140 attaches.

The example instrument fiducial array 140 is discussed in greater detail below, along with several other example fiducial arrays that have increased visibility. For now, consider that the instrument fiducial array 140 comprises an array structure 252 defining a plurality of reflector receptacles (e.g. reflector receptacles 254 and 256). Within each reflector receptacle is a reflector or fiducial (e.g., fiducials 258 and 260 associated with reflector receptacles 254 and 256, respectively). The array structure 252 further defines a portion designed and constructed to mate with the slot 250 of the array connector 230, the portion termed herein the fiducial connector 262. The example instrument fiducial array 140 and the fiducials are rigidly attached to the cutter 134. Any movement as between the handpiece 136 and the sleeve connector 202 and/or mechanical resection device 200 is extraneous and independent of the tracking system.

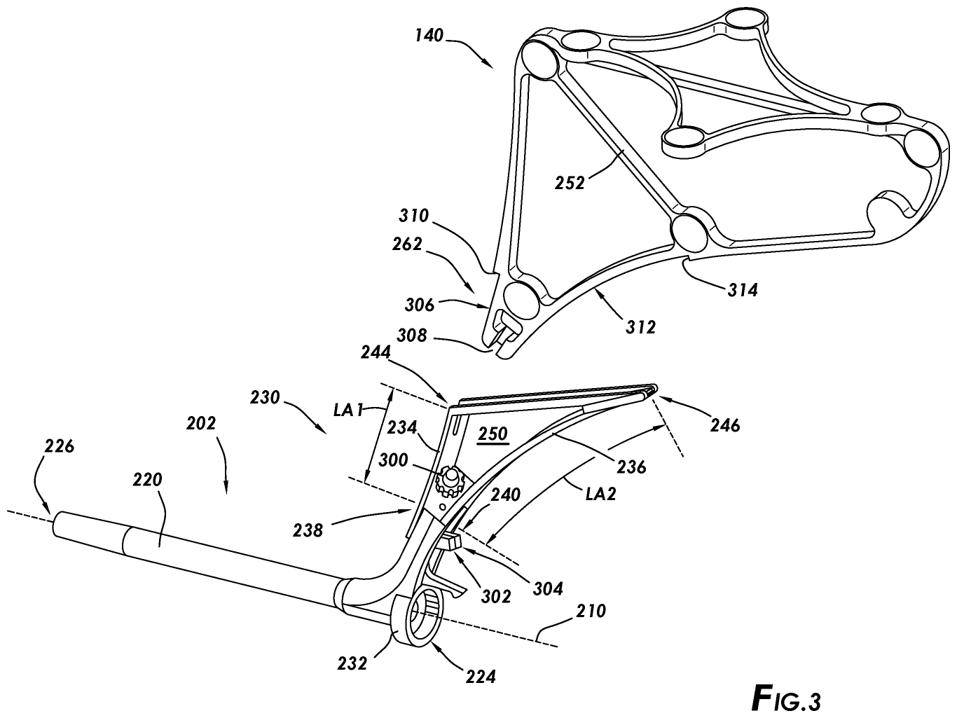

FIG. 3 shows an exploded perspective view of the example sleeve connector 202 and the example instrument fiducial array 140. In particular, and considering the array connector 230, the first arm 234 defines a length LA1 measured from the proximal end 238 to the distal end 244. The second arm 236 defines a length LA2 measured from the proximal end 240 to the distal end 246. In accordance with some examples, the length LA1 of the first arm 234 is less than half the length LA2 of the second arm 236. Moreover, and as shown, a separation between the first arm 234 and the second arm 236, measured across the slot 250, increases with increasing distance from the proximal end 224 of the sleeve 220. Equivalently stated, the separation between the first arm 234 and the second arm 236 across the slot 250 increases with increasing distance with respect to the longitudinal central axis 210. In the example of FIG. 3, the second arm 236 defines a radius of curvature with the center of the radius of curvature outside the slot 250, and the center of the radius of curvature residing between the array arm 228 and the longitudinal central axis 210. In other cases, however either or both of the first and second arms 234 and 236 may be straight, yet still define the increasing separation with distance. Finally, at the proximal end 238 of the first arm 234 and the proximal end 240 the second arm 236 resides a latch 300. The latch 300 is designed and constructed to hold the instrument fiducial array 140 in place when the fiducial connector 262 of the instrument fiducial array 140 is coupled within array connector 230.

Also better visible in FIG. 3 are latches defined by the sleeve connector 202, the latches used to hold the sleeve connector 202 in fixed axial position along the longitudinal central axis 210. In particular, for reasons that will become clearer based on the discussion below, the example system comprises latches 302 and 304 in operational relationship to the receptacle 232. In the view of FIG. 3, the release buttons are both visible, but only the latch arm for latch 302 is visible owing to different lengths of the latch arms (discussed more below).

Still referring to FIG. 3, the example instrument fiducial array 140 again comprises the array structure 252. The array structure 252 defines the fiducial connector 262, and the fiducial connector 262 is designed and constructed to telescope within the slot 250. In particular, the fiducial connector 262 defines a first mating surface 306 that extends from the distal tip 308 to a shoulder region 310. Similarly, the fiducial connector 262 defines a second mating surface 312 that extends from the distal tip 308 to a shoulder region 310. When the example instrument fiducial array 140 is coupled to the sleeve connector 202, the first mating surface 306 abuts an inside wall of the first arm 234, the second mating surface 312 abuts an inside wall of the second arm 236, and the shoulder regions 310 and 314 about the distal ends 244 and 246, respectively.

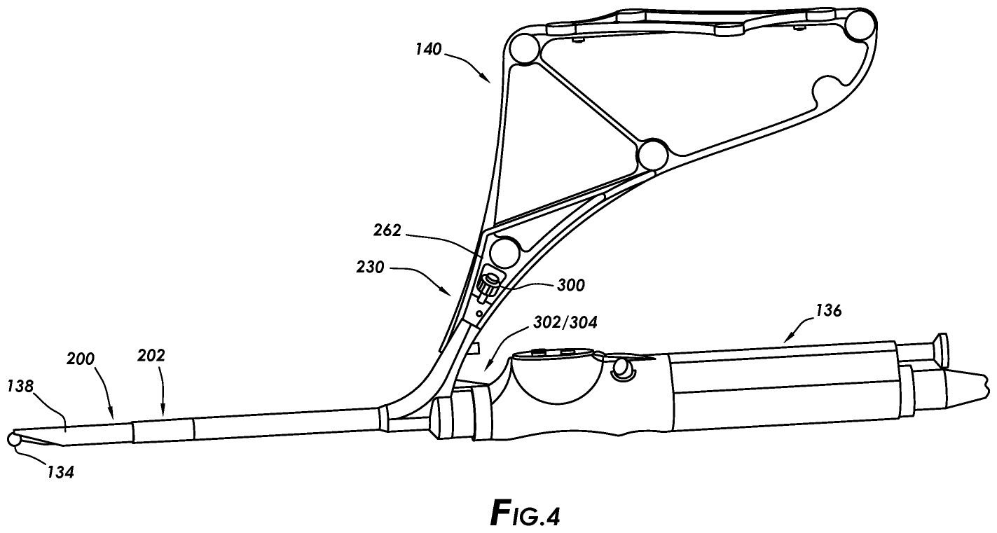

FIG. 4 shows a side elevation view of the resection instrument system 106 in assembled form. In particular, FIG. 4 shows the handpiece 136 with the mechanical resection device 200 operatively coupled to the handpiece 136 by having the inner hub 216 (not visible, FIG. 2) and stationary outer hub 214 (not visible, FIG. 2) telescoped within the receptacle 208 (not visible, FIG. 2). The sleeve connector 202 is telescoped over and concentrically arranged around the elongate outer tube 138 of the mechanical resection device 200, and one of the latches 302/304 is latched to the stationary outer hub 214 to hold the sleeve connector 202 in place axially relative to the elongate outer tube 138. Further, the instrument fiducial array 140, and specifically the fiducial connector 262, is in a mating relationship with the array connector 230 such the instrument fiducial array 140 is held rigidly in place relative to the sleeve connector 202 and thus the cutter 134. The specification now turns to a more detailed description of the mating connection between the fiducial connector 262 and the array connector 230.

FIG. 5 shows a partial cross-sectional view of the array connector mated with the fiducial connector of the instrument fiducial array. In particular, visible in FIG. 5 is a portion of the array connector 230 and a portion of the instrument fiducial array 140 including the fiducial connector 262. In the view of FIG. 5, the fiducial connector 262 is telescoped within the slot 250 and is in mating relationship with the array connector 230. In order to hold the instrument fiducial array 140 rigidly within the array connector 230, the array connector 230 conceptually defines at least three points of contact that form connections and that become more rigid with increasing holding force. In particular, better visible in FIG. 5 is the intersection 500 between the first arm 234 and the second arm 236. The example intersection 500 defines a radius of curvature with a center that resides within the slot 250. Other shapes form the intersection are possible, including a V shape.

Now consider the first arm 234, and in particular the distal end 244 of the first arm 234. The distal end 244 of the first arm 234 defines an abutment surface 502. In the cross-sectional view of FIG. 5, the example abutment surface 502 defines a surface having an apex at the distal end of the surface, and the surface becoming wider along the first arm 234 toward the proximal end 238. More particularly still, the example abutment surface 502, in the cross-sectional view of FIG. 5, is a curved surface. The fiducial connector 262 likewise defines the shoulder region 310. In example embodiments, in cross-section the shoulder region 310 is complementary of the abutment surface 502. Thus, the example shoulder region 310 defines a trough designed and constructed to mate with the abutment surface 502. Much like the intersection 500, the combination of the abutment surface 502 and the shoulder region 310 are designed and constructed to create a connection that becomes more rigid with increasing holding force. Other cross-sectional shapes for the abutment surface 502 and shoulder region 310 are possible, including a V shape for the abutment surface 502 and a corresponding V-shaped trough for the shoulder region 310. The example of FIG. 5 shows the fiducial connector 262 abutting the first arm 234 from the abutment surface 502 to the intersection 500. However, in other cases the fiducial connector 262 may abut the first arm 234 only at the abutment surface 502 and intersection 500.

Now consider the second arm 236, and in particular the distal end 246 of the second arm 236. The distal end 246 of the second arm 236 defines an abutment surface 504. In the cross-sectional view of FIG. 5, the example abutment surface 504 defines a surface having an apex at the distal end of the surface, and the surface becoming wider along the second arm 236 toward the proximal end 240. More particularly still, the example abutment surface 504, in the cross-sectional view of FIG. 5, is a curved surface. The fiducial connector 262 likewise defines the shoulder region 314. In example embodiments, the shoulder region 314 is complementary of the abutment surface 504. Thus, the example shoulder region 314 defines a trough designed and constructed to mate with the abutment surface 504. As before, the combination of the abutment surface 504 and the shoulder region 314 are designed and constructed to create a connection that becomes more rigid with increasing holding force. Other cross-sectional shapes for the abutment surface 504 and shoulder region 314 are possible, and the cross-sectional shapes as between the abutment surface 504 and the abutment surface 502 of the first arm 234 need not be the same. The example of FIG. 5 shows the fiducial connector 262 abutting the second arm 236 from the abutment surface 504 to the intersection 500. However, in other cases the fiducial connector 262 may abut the second arm 236 only at the abutment surface 504 and intersection 500. That is, in some cases the three points of connection between the fiducial connector 262 and the array connector 230 (i.e., abutment surface 502, intersection 500, and abutment surface 504 are sufficient to hold the instrument fiducially array 140 to the sleeve connector 202.

Still referring to FIG. 5, the array connector 230 further comprises the latch 300 designed and constructed to impart or provide a holding force to the fiducial connector 262 to hold fiducial connector 262 in the mating relationship with the slot 250. The example latch 300 comprises a shaft 506. A distal end of the shaft 506 comprises external threads and is in operational relationship with a nut 508 having internal threads. The proximal end of the shaft 506 defines a through bore 510 aligned with a through bore 512 through the array connector 230. The through bore 510, the through bore 512, and a hinge pin 514 form a hinge 516 that enables the shaft 506 and nut 508 to rotate in and out of the fiducial connector 262. In the view of the FIG. 5, the shaft 506 and nut 508 rotate out of the fiducial connector 262 by rotating in the direction of the viewer. In order to accommodate the latch 300, the example fiducial connector 262 defines a slot 518 and a through bore 520. When the latch 300 is in operational relationship with the instrument fiducial array 140, the shaft 506 is disposed in the slot 518, and the nut 508 is disposed in the through bore 520. By turning the nut 508, a proximal or bottom surface of the nut 508 abuts an inside surface of the through bore 520 which imparts a force to the fiducial connector 262 tending to hold the fiducial connector 262 within the slot 250. Other types of latches may be equivalently used, including any of a variety of draw latches, tension latches, and over-center latches.

Returning briefly to FIG. 4. In FIG. 4, the latch 300 is shown in mid-position between a fully latched configuration (such as shown in FIG. 5) and a fully unlatched configuration such that the instrument fiducial array 140 may be removed from the sleeve connector 202. In some cases, the latching force provided by the latch 300, in combination with the interactions of the fiducial connector 262 with the various physical features of the array connector 230 (e.g., the intersection 500 and the abutment surfaces 502/504) may be sufficient to rigidly hold the instrument fiducial array 140 relative to the sleeve connector 202 and thus the mechanical resection device 200. However, in yet still other cases, the mechanical connection between the fiducial connector 262 and the array connector 230 may be further improved by use of features designed and constructed to further limit movement of the instrument fiducial array 140 in directions transverse to a plane defined by the arms of the array connector 230. That is, and in the view of FIG. 4, the arms of the array connector 230 define a plane that is parallel to the plane of page. Directions transverse to the plane of the page are directions into and out of the plane of the page.

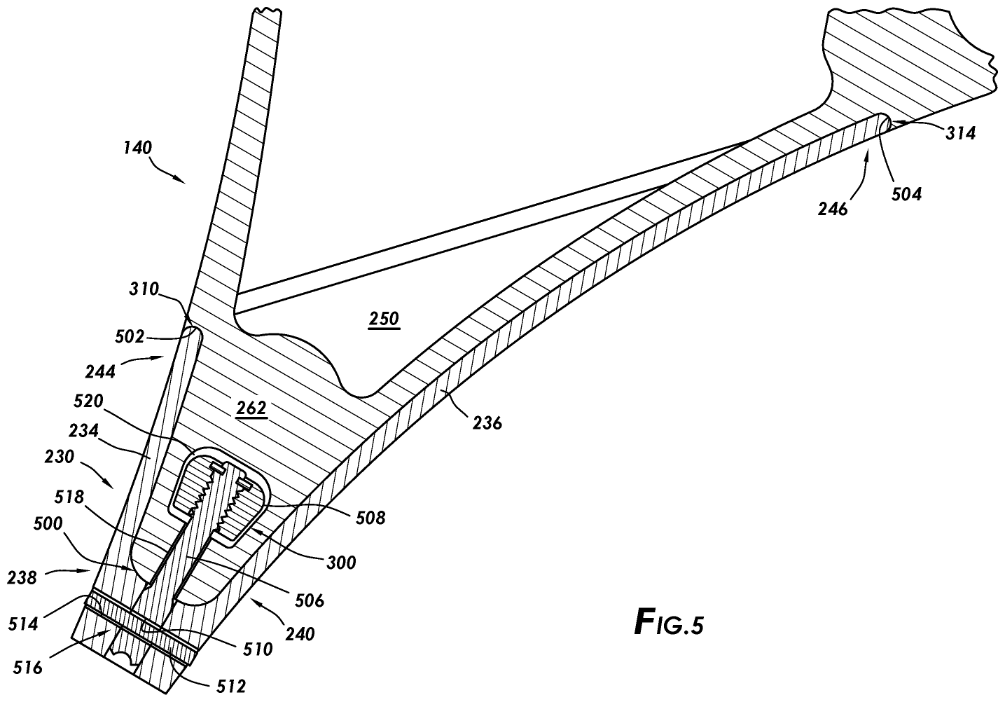

FIG. 6 shows a partial front perspective view of the connection between the array connector 230 and the fiducial connector 262. In particular, visible in FIG. 6 is the distal end 244 of the first arm 234, a portion of the second arm 236 in the background, and the support members 242 and 248 coupled between the distal ends of the first and second arms 234 and 236. In the example, a notch 600 is defined on the distal end 244 of the first arm 234. The example notch 600 defines a channel with closed bottom and an open top, the open top is defined by the distal end 244 of the first arm 234 and/or the support members 242 and 248. In the example, an imaginary line from the closed bottom to the open top of the notch 600 is parallel to the length LA1 (FIG. 3) of the first arm 234. In the example, the fiducial connector 262 defines a width (measured perpendicular to the length LA1 of the first arm) selected to form an interference fit within the notch 600. In particular, the width of the notch 600 (measured perpendicular to the length LA1 of the first arm) and the width of the fiducial connector 262 are designed and selected such that, during insertion of the fiducial connector 262 into the notch 600, the side walls of the notch 600 elastically deform symmetrically, thus maintaining centered contact between the instrument fiducial array 140 and the array connector 230. In some cases, and a shown, at additional notch 610 may be included, the additional notch 610 disposed proximally from the notch 600. The additional notch 610 has a closed bottom and an open top where the open top of the additional notch 610 intersects the otherwise closed bottom of notch 600. The additional notch may aid in the elastic deformation.

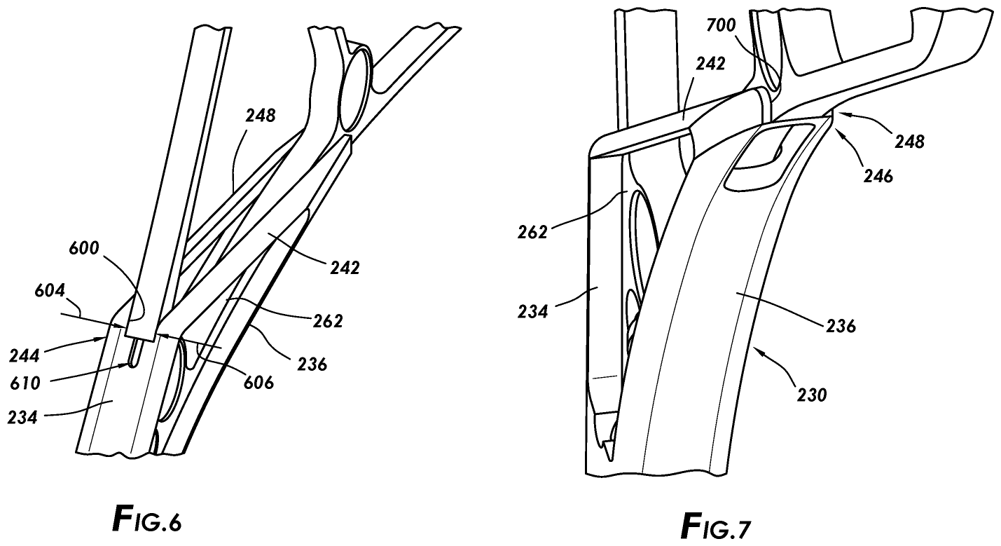

FIG. 7 shows a partial back perspective view of the connection between the array connector 230 and the fiducial connector 262. In particular, visible in FIG. 7 is the distal end 246 of the second arm 236, a portion of the first arm 234 in the background, and the support members 242 coupled between the distal end of the first and second arms 234 and 236. In the example, a notch 700 is defined on the distal end 246 of the second arm 236. The example notch 700 defines a channel with a closed bottom and an open top. For the example notch 700, the open top is defined by upper surfaces of the support members 242 and 248. The fiducial connector 262 defines a width selected to form an interference fit within the notch 700. In particular, the width of the notch 700 (measured perpendicular to the length LA2 of the second arm 236) and the width of the fiducial connector 262 are designed and selected such that, during insertion of the fiducial connector 262 into the notch 700, the sidewalls of the notch 700 elastically deformed symmetrically, thus increasing opposing forces applied against the instrument fiducial array 140.

The notch 600 and the notch 700 operating on the overall width of the fiducial connector 262, are merely examples of having a portion of the fiducial connector 262 reduce clearance fits and thus looseness between the instrument fiducial array 140 and the sleeve connector 202. In yet still other examples, the notch 600 and/or the notch 700 may be designed and constructed to be elastically deformed by a smaller ridge defined by the fiducial connector, rather than by the overall width of the fiducial connector 262. The specification now turns to a more detailed description of the latches 302 and 304 that hold the sleeve connector 202 on the mechanical resection device 200.

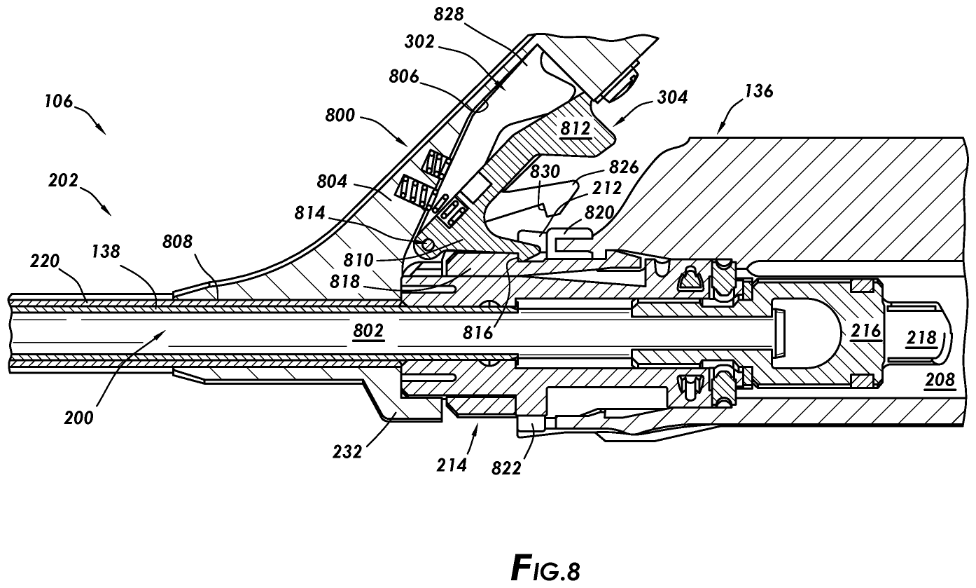

FIG. 8 shows a partial cross-sectional view of a resection instrument system 106. The cut for the view of FIG. 8 not along the longitudinal central axis of the elongate outer tube 138; rather, the cut is offset toward the viewer of FIG. 8 in order to show the latches 302 and 304 in better detail. Working from left to right in the figure, FIG. 8 shows the sleeve connector 202. The sleeve connector 202 is telescoped over and concentrically arranged around the mechanical resection device 200. The mechanical resection device 200 includes the elongate outer tube 138, the stationary outer hub 214, and the inner hub 216. The inner hub 216 is coupled to an inner tube 802. The stationary outer hub 214 and the inner hub 216 are shown telescoped within the receptacle 208 of the handpiece 136, but the internal connection to the drive tang 218 of the inner hub 216 to a rotating connector is omitted so as not to further complicate the figure.

The sleeve connector 202 defines the sleeve 220. On the proximal end of the sleeve 220 is a transition piece 800 disposed between the sleeve 220 and the array connector 230 (not visible). The example transition piece 800 defines the receptacle 232 as well as a transition arm 804. The example transition arm 804 projects away from the proximal end of the sleeve 220, and the distal end of the transition arm 804 couples to and/or defines the proximal ends 238 and 240 (FIG. 2) the first arm 234 and second arm 236 (FIG. 2), respectively. The example transition piece 800 further defines a latch recess 806 in which the latches 302 and 304 at least partially reside. In the example shown, the transition piece 800 is shown as a separate element defining a through bore 808 telescoped over and concentrically arranged over the outside diameter of the sleeve 220, in some cases forming a friction fit. However, in other cases the combination of the sleeve 220 and transition piece 800 may be an integrated component (e.g., a single component). In yet still other cases, the transition piece 800 may form a slidable or rotatable fit with the sleeve 220 such that the rotational orientation of the sleeve 202 and transition piece 800 relative to the longitudinal central axis of the sleeve may be adjustable.

Latch 304 defines a latch arm 810, a release arm 812, and a hinge 814. The latch arm 810 defines a length from the center of rotation of the hinge 814 to the latch surface 816. The length from the center of rotation of the hinge 814 to the latch surface 816 is designed and constructed to latch over a feature of the stationary outer hub 214, an in particular the annular alignment ridge 818. The example annular alignment ridge 818 is an annular ridge that at least partially circumscribes the stationary outer hub 214. To release the sleeve connector 202 from the mechanical resection device 200, the user presses the release arm 812, which rotates the latch 304 about the hinge 814 to raise the latch arm 810 and latch surface 816 away from the alignment ridge 818.

In accordance with example systems, the mechanical resection device 200 couples to the handpiece 136 in at least two rotational orientations relative to the longitudinal central axis of the elongate outer tube 138. In the example system, the stationary outer hub 214 defines an alignment ridge 820 on a first side of the stationary outer hub 214, and a second alignment ridge 822 on an opposite side of the stationary outer hub 214. Stated otherwise, the alignment ridge 820 extends outward in a radial direction relative to the longitudinal central axis of the elongate outer tube 138, and the alignment ridge 822 extends outward in a radial direction opposite the alignment ridge 820. The alignment ridge 820 serves double duty, also acting as the release button to decouple the stationary outer hub 214 from the receptacle 208. The rotational orientation of the alignment ridges 820 and 822 relative to the slots 212 and 824 defined by the receptacle 208 of the handpiece 136 define the two example rotational orientations of the mechanical resection device 200. In the example of FIG. 8, the alignment ridge 820 is in operational relationship to the slot 212 on the upper side of the handpiece 136, and in the rotational orientation shown the latch 304 latches the sleeve connector 202 to the mechanical resection device 200 by way of the annular alignment ridge 818. However, if the mechanical resection device 200 is coupled to the handpiece 136 such that the alignment ridge 822 is within the slot 212, or the mechanical resection device 200 is as shown in FIG. 8 but the sleeve connector 202 is rotated 180 degrees about the longitudinal central axis of the elongate outer tube 138, the annular alignment ridge 818 is not available for latching by the latch 304. In that case, latch 302 may be used to latch the sleeve connector 202 to the mechanical resection device 200.

Still referring FIG. 8, latch 302 defines a latch arm 826, a release arm 828, and share a hinge 814 with the latch 304. The latch arm 826 defines a length from the center of rotation of the hinge 814 to a latch surface 830, and the length of the latch arm 826 is longer than a length of the latch arm 810 correspondingly measured. The length from the center of rotation of the hinge 814 to the latch surface 830 is designed and constructed to latch over a feature of the stationary outer hub 214, and in particular the alignment ridge 822. In view of FIG. 8, however, the mechanical resection device 200 is coupled to the handpiece 136 in a rotational orientation in which the alignment ridge 822 is 180 angular degrees away from the location of the latch arm 826 and latch surface 830. However, when the mechanical resection device 200 is coupled such that alignment ridge 822 is at the top of the view of FIG. 8, or the mechanical resection device 200 is as shown in FIG. 8 but the sleeve connector 202 is rotated 180 degrees about the longitudinal central axis of the elongate outer tube 138, the latch surface 830 of the latch arm 826 will latch to the alignment ridge 822.

FIG. 9 shows an example sleeve connector 202. In particular, visible in FIG. 9 are the sleeve 220, the transition piece 800, and the array connector 230. The various examples discussed to this point have assumed a slip fit between the inside diameter of the sleeve 220 and the outside diameter of the elongate outer tube 138 (FIG. 1), the slip fit with sufficient tolerances to reduce or eliminate movement that shifts the longitudinal central axis of the sleeve 220 relative to the longitudinal central axis of the elongate outer tube 138, and which axes should be coaxial when the sleeve 220 is concentrically arranged around the elongate outer tube 138. However, in other cases the tolerances for the slip fit may enable slight movement of the relative axes, and thus in additional embodiments the sleeve 220 includes various springs to help center the sleeve 220 around the elongate outer tube 138, and possibly also to provide additional forces to help hold the sleeve at designated axial positions relative to the shared longitudinal central axis with the mechanical resection device.

In particular, FIG. 9 shows a set of springs defined by and through the sleeve 220. Example spring 900 is a leaf spring with a fixed end 902 and a free end 904, where the free end 904 has an unbiased or rest orientation disposed within the internal volume of the sleeve 220. The example spring 900 may be construed in any suitable way, such as by laser cutting the sleeve 220 and then bending the spring element inward. In example embodiments, a plurality of such springs may be placed along and around the sleeve 220. For example, sleeve 220 comprises additional springs 906 and 908 at different and displaced axial positions along the sleeve 220. In additional to, or in place of, having springs at varying axial positions, the springs may be at varying radial positions around the sleeve 220 relative to the longitudinal central axis of the sleeve 220. For example, two or more additional springs may be located at the same axial position as spring 900, but at different radial locations (and thus are not visible in FIG. 9). Nevertheless, the springs may be designed, constructed, and located to increase a holding force of the sleeve connector 202 to the elongate outer tube 138, and/or to provide a net force tending to center the sleeve 220 concentrically around the elongate outer tube 138 of the mechanical resection device 200.

Still referring to FIG. 9, the example sleeve connector 202 further comprises additional spring elements at the distal end 222 of the sleeve 220. In particular, the distal end 222 of the sleeve 220 defines a plurality of springs arranged annularly around the distal end 222 of the sleeve 220. In the view of FIG. 9, three such springs 910, 912, and 914 are fully or partially visible. Referring to spring 910 as representative, the spring 910 defines a fixed end 916 and a free end 918. The springs 910, 912, and 914 may be designed and constructed to increase a holding force of the sleeve connector 202 to the elongate outer tube 138, and/or to provide a net force tending to center the sleeve 220 concentrically around the elongate outer tube 138 of the mechanical resection device 200. The specification now turns to various examples of fiducial arrays, such as instrument fiducial array 140.

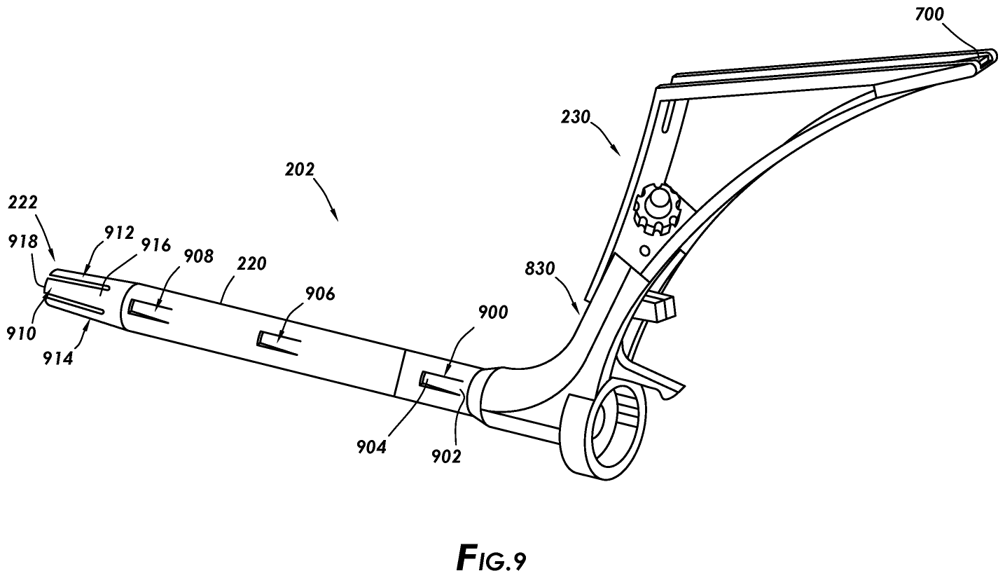

FIG. 10 shows a perspective view of the example instrument fiducial array 140. The example instrument fiducial array 140 is designed and constructed to couple to the sleeve connector 202 and thus the mechanical resection device 200. The arrangement of the fiducials, discussed in greater detail below, provides increased visibility to a stereoscopic camera through greater range of motion in relation to related-art devices. The arrangement of the fiducials may find application with respect to any device or structure to be tracked during surgery, such as a fiducial array associated with a femur, a fiducial array associated with the acetabulum, or fiducial arrays associated with other surgical instruments.

The example instrument fiducial array 140, and in particular the array structure 252, defines an upper set of reflector receptacles 1000, 1002, 1004, and 1006. In example cases the array structure 252 is made of a metallic material suitable for sterilization within an autoclave, but any suitably rigid material that can withstand an autoclave may be used. In yet still other cases, the array structure 252 is a disposable element, and thus need not be made of material suitable for sterilization in an autoclave (e.g., a rigid plastic). Within each reflector receptacle is placed respective reflector or fiducial 1008, 1010, 1012, and 1014. The upper set of reflector receptacles 1000, 1002, 1004, and 1006 define and reside in an upper plane. Stated equivalently, the example fiducials 1008, 1010, 1012, and 1014 each have reflective surfaces, and the reflective surfaces define and reside in the upper plane. The fiducials 1008, 1010, 1012, and 1014 define a pattern based on the spacing and separation within the upper plane. During operative procedures the procedure controller 108 (FIG. 1), using the stereoscopic camera 110 (FIG. 1), may recognize the pattern as the fiducials of the upper plane of the instrument fiducial array 140 based on the pattern. The procedure controller 108 may determine the orientation of the attached device within the three-dimensional space of the surgical room, and for the example instrument fiducial array 140 the underlying device would be the mechanical resection device 200 (FIG. 2) and specifically the cutter 138 (FIG. 2). When two planes are visible to the tracking camera, a software algorithm picks one plane as the actively-tracked plane. In the example using perpendicular (fiducial) planes, the fiducials are selected to have reflectance angles (discussed more below) so that there is no dead zone of no reflectance as the camera shifts from one plane to the next.

Still referring to FIG. 10, the example instrument fiducial array 140, and particularly the array structure 252, further defines the first side set of reflector receptacles 254, 256, 1016, and 1018. Within each reflector receptacle is placed respective fiducial 258, 260, 1020 and 1022. The first side set of reflector receptacles 254, 256, 1016, and 1018 define and reside in a first side plane. Stated equivalently, the example fiducials 258, 260, 1020 and 1022 each have reflective surfaces that define and reside in the first side plane. In the example system, the first side plane forms a right angle or is perpendicular to (i.e., normal to) the upper plane, and the first side plane intersects the upper plane between at least two fiducials. In other cases, however, the fiducials of the upper plane may all reside to one side of the intersection. The fiducials 258, 260, 1020 and 1022 define a pattern based on the spacing and separation within the first side plane, and the pattern of the fiducial in the first side plane is different than the pattern of the fiducials in the upper plane. During operative procedures, the procedure controller 108 (FIG. 1), using the stereoscopic camera 110, may recognize the pattern as the fiducials on the first side plane of the instrument fiducial array 140 based on the pattern. The procedure controller 108 may determine the orientation of the attached device within the three-dimensional space of the surgical room.

In the example instrument fiducial array 140, reflector receptacles 254, 256, and 1016 perform double duty in the sense that, in the perspective view of FIG. 10, the opposite side of the reflector receptacles 254, 256, and 1016 are also members in a second side set of reflector receptacles. Thus, the fiducials 258, 260, and 1020 have reflective surfaces on both sides of the fiducial, and thus the opposite sides of fiducials 258, 260, and 1020 define and reside in the second side plane. In the example system, the second side plane is parallel to the first side plane, the second side plane forms a right angle or is perpendicular to the upper plane, and the second side plane intersects the upper plane between at least two fiducials of the upper plane. In other cases, however, the fiducials of the upper plane may all reside to one side of the intersection. So as to differentiate the pattern of fiducials on the second side plane from the fiducials on the first side plane, in the example system a fiducial 1022 participates only in the first side plane, and a fiducial associated with reflector receptacle 1024 participates only in the second side plane. In particular, the example array structure 252 further defines the reflector receptacle 1024. The reflector receptacle 1024 has disposed therein a fiducial that is not visible in the view of FIG. 10. However, the fiducial in the reflector receptacle 1024, along with the opposite sides of fiducials 258, 260, and 1020 define a pattern based on the spacing and separation within the second side plane, and the pattern of the fiducial in the second side plane is different than both the pattern of the fiducials in the upper plane and the pattern of fiducials in the first side plane. During operative procedures the procedure controller 108 (FIG. 1), using the stereoscopic camera 110 (FIG. 1), may recognize the pattern as the fiducials on the second side plane of the instrument fiducial array 140 based on the pattern. The procedure controller 108 may determine the orientation of the attached device within the three-dimensional space of the surgical room.

Still referring to FIG. 10, but stated broadly, in example systems a fiducial array may comprise a first set of reflector receptacles, the first set of reflector receptacles define and reside in a first plane. A first set of fiducials may be disposed one each in each of the first set of reflector receptacles. The fiducial array may further comprise a second set of reflector receptacles, the second set of reflector receptacles define and reside in a second plane. The second set of fiducials may be disposed one each in each of the second set of reflector receptacles. The fiducial array may further comprise a third set of reflector receptacles, the third set of reflector receptacles define and reside in a third plane. The third set of fiducials may be disposed one each in each of the third set of reflector receptacles. The underlying array structure holds the first set of reflector receptacles, the second set of reflector receptacles, and the third set of reflector receptacles in a fixed orientation.

As will be discussed in more detail below, the viewing angle for any particular fiducial is bounded by a conic frustum having the smaller diameter about equal to the diameter of the reflective face of the fiducial, the conic frustum extending outward along a central axis normal to the reflective face of the fiducial, and a side wall forming an angle of 45 angular degrees or more to the central axis. It follows that the each set of fiducials in a plane has a viewing angle of about 90 angular degrees. Having a fiducial array with three planes, the stereoscopic camera 110 (FIG. 1) should have the ability to “see” at least one of the sets of fiducials at any one time, and in some orientations the stereoscopic camera 110 may see two of the sets of fiducials at any one time. Given that the location of the fiducial array within the three-dimensional space of the surgical room may be determined from any one of the sets of fiducials alone, the procedure controller 108 may thus determine the location of the fiducial array, and the underlying attached device, in any orientation of the attached device. Moreover, in having differing patterns of fiducials as between the first side plane and the second side plane, the procedure controller 108 (FIG. 1) may be able to determine which direction the tracked device is facing.

The example instrument fiducial array 140 defines three planes—an upper plane, a first side plane perpendicular to the upper plane, and a second side plane perpendicular to the upper plane and parallel to the first side plane. However, the arrangement of the planes need not be perpendicular and/or parallel to achieve the increased viewing range by the stereoscopic camera 110 (FIG. 1). The discussion thus turns to fiducial arrays having fiducials residing in three planes where those planes are not parallel, and where any two of the planes are not necessarily perpendicular.

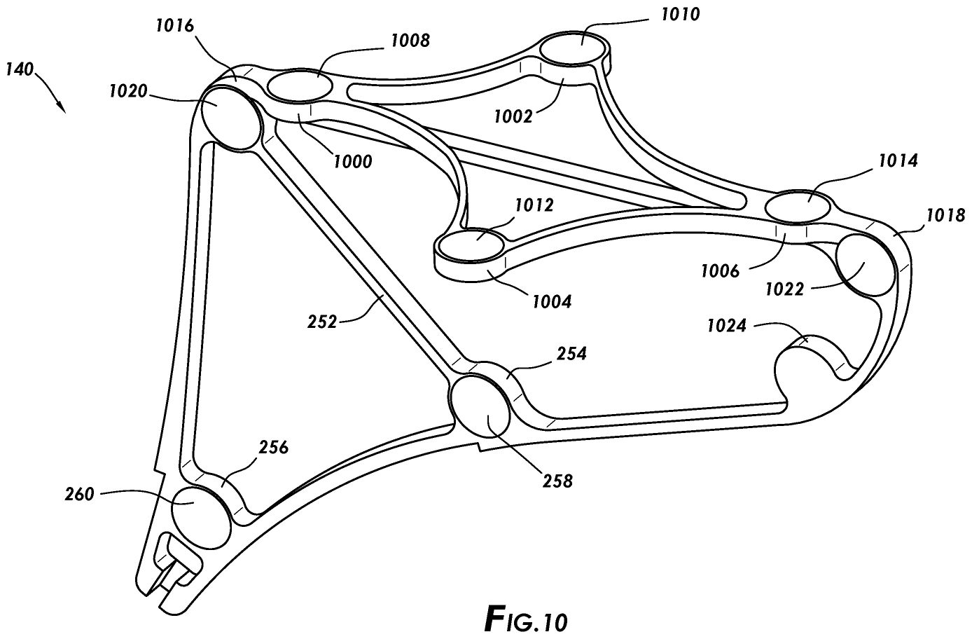

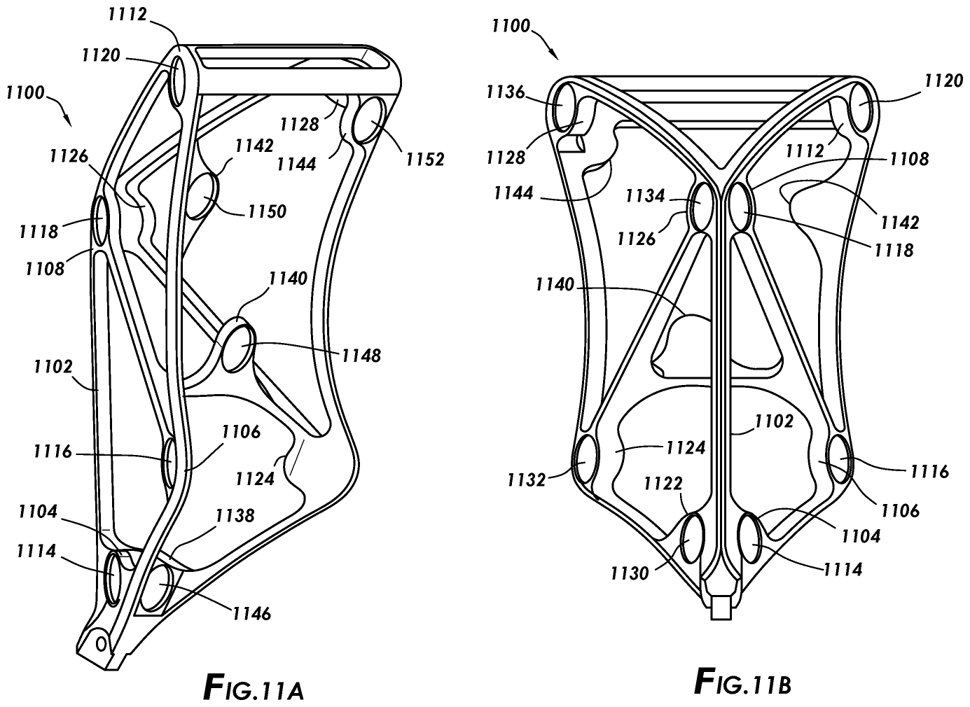

FIG. 11A shows a back perspective view of another example fiducial array 1100, and FIG. 11B shows a front elevation view of the example fiducial array 1100. Referring simultaneously to FIGS. 11A and 11B. In particular, the example fiducial array 1100 comprises an array structure 1102. In many cases, the array structure 1102 is a metallic material suitable sterilization in an autoclave, but any suitably rigid material that can withstand sterilization may be used. The example fiducial array 1100, and in particular the array structure 1102, defines first set of reflector receptacles 1104, 1106, 1108, and 1112. Within each reflector receptacle is placed respective fiducial 1114, 1116, 1118, and 1120. The first set of reflector receptacles 1104, 1106, 1108, and 1112 define and reside in a first plane. Stated equivalently, the example fiducials 1114, 1116, 1118, and 1120 each have a reflective surface, and the reflective surfaces define and reside in the first plane. The fiducials 1114, 1116, 1118, and 1120 define a pattern based on the spacing and separation within the first plane. During operative procedures the procedure controller 108 (FIG. 1), using the stereoscopic camera 110 (FIG. 1), may recognize the pattern as the fiducials of the first plane of the fiducial array 1100 based on the pattern. The procedure controller 108 may determine the orientation of the attached device within the three-dimensional space of the surgical room.

The example fiducial array 1100, and in particular the array structure 1102, further defines second set of reflector receptacles 1122, 1124, 1126, and 1128. Within each reflector receptacle is placed respective fiducial 1130, 1132, 1134, and 1136. The second set of reflector receptacles 1122, 1124, 1126, and 1128 define and reside in a second plane. Stated equivalently, the example fiducials 1130, 1132, 1134, and 1136 each have a reflective surface, and the reflective surfaces define and reside in the second plane. The fiducials 1130, 1132, 1134, and 1136 define a pattern based on the spacing and separation within the second plane. During operative procedures the procedure controller 108 (FIG. 1), using the stereoscopic camera 110 (FIG. 1), may recognize the pattern as the fiducials of the second plane of the fiducial array 1100 based on the pattern. The procedure controller 108 may determine the orientation of the attached device within the three-dimensional space of the surgical room.

The example fiducial array 1100, and in particular the array structure 1102, further defines third set of reflector receptacles 1138, 1140, 1142, and 1144. Within each reflector receptacle is placed respective fiducial 1146, 1148, 1150, and 1152. The third set of reflector receptacles 1138, 1140, 1142, and 1144 define and reside in a third plane. Stated equivalently, the example fiducials 1146, 1148, 1150, and 1152 each have a reflective surface, and the reflective surfaces define and reside in the third plane. The fiducials 1146, 1148, 1150, and 1152 define a pattern based on the spacing and separation within the third plane. During operative procedures the procedure controller 108 (FIG. 1), using the stereoscopic camera 110 (FIG. 1), may recognize the pattern as the fiducials of the third plane of the fiducial array 1100 based on the pattern. The procedure controller 108 may determine the orientation of the attached device within the three-dimensional space of the surgical room.

Still referring to FIGS. 11A and 11B, in the example systems the fiducial array 1100 defines the three planes. The first plane and second plane intersect, and the acute angle between them is less than 90 angular degrees. The second plane and third plane intersect, and the acute angle between them is less than 90 angular degrees. It follows that the third plane and first plane intersect, and the acute angle between them is less than 90 angular degrees. Considered together, the three planes define an internal volume. In yet still other cases, one of angles between the planes may form a right angle measured through the internal volume.

In some example cases, the planes defined by the reflector receptacles and/or the fiducials may intersect outside an area bounded by the reflector receptacles and/or the fiducials. For example, the first plane and the second plane of the example fiducial array 1100 intersect outside the boundary of their respective reflector receptacles and/or fiducials. In other cases, the planes defined by the reflector receptacles and/or the fiducials may intersect an abutting plane within an area bounded by the reflector receptacles and/or the fiducials. In the example of fiducial array 1100, the third plane (defined, for example, by fiducials 1146, 1148, 1150, and 1152) intersects the first plane between fiducials 1116 and 1118. Similarly, the third plane intersects the second plane between the fiducials 1132 and 1134. Thus, any suitable arrangement for intersection of the planes may be used.

As before, by having a fiducial array with three planes as illustrated by FIGS. 11A and 11B, the stereoscopic camera 110 (FIG. 1) may have the ability to “see” at least one of the sets of fiducials at any one time, and in some orientations the stereoscopic camera 110 may see two of the sets of fiducials at any one time. Given that the location of the fiducial array within the three-dimensional space of the surgery may be determined from any one of the sets of fiducials alone, the procedure controller 108 may thus determine the location of the fiducial array, the underlying attached device, in any orientation of the attached device. Moreover, in having a differing patterns of fiducials as between the first side plane and the second side plane (e.g., different in a mirror-image sense), the procedure controller 108 (FIG. 1) may be able to which direction the tracked the device is facing. The specification now turns to a description of an example fiducial.

FIG. 12 shows a perspective view of an example fiducial 1200. The example fiducial 1200 may be representative of any of the previously discussed fiducials in the various fiducial arrays. In particular, the example fiducial 1200 comprises a body 1202 defining a front surface 1204, a back surface (not visible in FIG. 12), and a central axis 1206. In example cases, the front surface 1204 is parallel to the back surface. Disposed on the front surface 1204 is a front reflective surface 1208, and in some cases the back surface incudes a back reflective surface. The example fiducial 1200 is a symmetric device front to back, and thus a back perspective view would be a mirror image of FIG. 12. So as not to unduly lengthen the specification, the back perspective view is omitted.

The example fiducial 1200 defines a front annular latch system associated with front surface and a back annular latch system associated with the back surface. The example front annular latch system comprises four latch members 1210, 1212, 1214, and 1216. While four latch members are shown in FIG. 12, two or more latch members associated with each reflective surface may be used. Collectively the example four latch members 1210, 1212, 1214, and 1216 circumscribe the body 1202. As discussed in greater detail below, each latch member defines a hinge proximate the opposite surface, a cantilever projecting toward the associated surface, and a catch surface or annular latch surface. Referring to latch member 1212 as representative of the members of the front latch system, the example latch member 1212 defines a hinge 1218 proximate to the back surface of the fiducial 1200, a cantilever 1220 projecting toward the front surface 1204, and a catch surface or annular latch surface 1222 disposed at the distal end of the cantilever 1220. The annular latch surface 1222 defines a surface either co-planar with the front surface 1204, or in some cases parallel to the front surface 1204. Thus, each of the remaining latch members 1210, 1214, and 1216 each have a hinge, cantilever, and annular latch surface similar to latch member 1212, but at different radial locations relative to the central axis 1206.

The example back annular latch system comprises four latch members 1224, 1226, 1228, and 1230. While four latch members are shown in FIG. 12, two or more latch members associated with each reflective surface may be used. Collectively the example four latch members 1224, 1226, 1228, and 1230 circumscribe the body 1202. Each latch member defines a hinge proximate the front surface, a cantilever projecting toward the back surface, and an annular latch surface. Referring to latch member 1226 as representative of the members of the back latch system, the example latch member 1226 defines a hinge 1232 proximate to the front surface of the fiducial 1200, a cantilever 1234 projecting toward the back surface, and a catch surface or annular latch surface 1236 disposed at the distal end of the cantilever 1234. The annular latch surface 1236 defines a surface either co-planar with the back surface, or in some cases parallel to the back surface. Thus, each of the remaining latch members 1224, 1228, and 1230 each have a hinge, cantilever, and annular latch surface similar to latch member 1226, but at different radial locations relative to the central axis 1206.

Still referring to FIG. 12, the latch members of the front latch system and the back latch system are interspersed around the body 1202. Starting with latch member 1210 and working clockwise, latch member 1224 is disposed between latch members 1210 and 1212. Latch member 1212 is disposed between latch members 1224 and 1226. Latch member 1226 is disposed between latch members 1212 and 1214. And so on, with latch member 1210 residing between latch members 1230 and 1224.

In the example system, the front latch system has four latch members and the back latch system has four latch members. Given the interspersed relationship of the latch members and the number of latch members in each latch system, each latch members spans a circumferential distance of ⅛^{th} of the circumference of the latch members less the notch or gap width. For latch systems with three latch members (for a total of six latch members as between the front and back latch systems), each latch member spans a circumferential distance of ⅙^{th} of the circumference of the latch members less the notch or gap width.

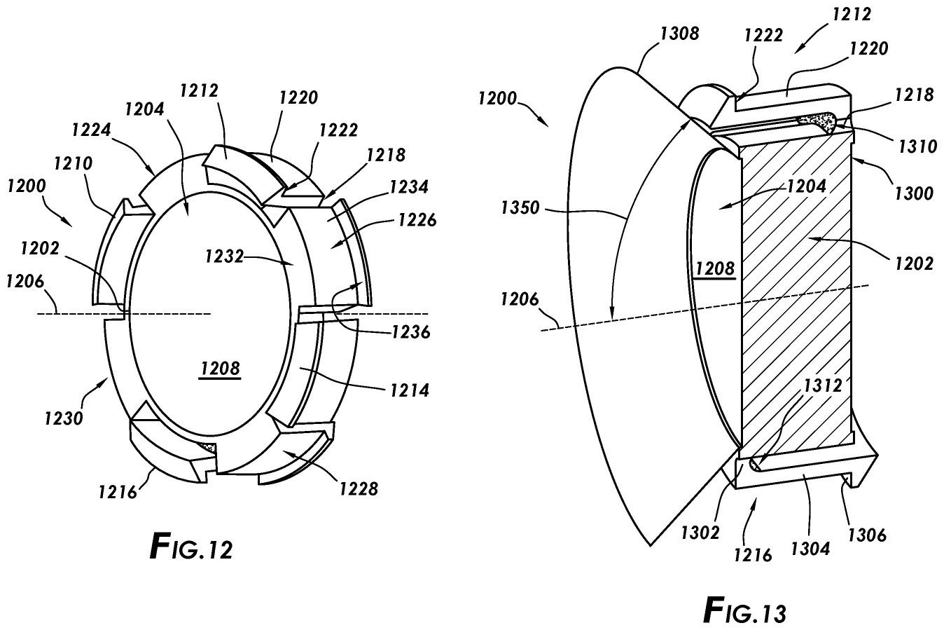

FIG. 13 shows a cross-sectional view of the example fiducial 1200. In particular, visible in FIG. 13 is the body 1202 defining the central axis 1206. In the view of FIG. 13, two of the latch members, one associated with the front surface and one associated with the back surface, are shown in cross-section. Referring initially to latch member 1212 of the front latch system. Better shown in FIG. 13 is the hinge 1218. The hinge 1218 in the example systems is constructed of the same material (e.g., polymeric material, such as plastic) that makes up the body 1202 of the fiducial 1200. Movement enabled by the hinge 1218 is created by elastic deformation of the underlying material. Moreover, the cross-sectional view of FIG. 13 shows the example hinge 1218 disposed adjacent to the back surface 1300. Extending or projecting toward the front surface 1204 is the example cantilever 1220, and on a distal end of the cantilever 1220 is the annular latch surface 1222.

Now referring to latch member 1216 of the back latch system. Better shown in FIG. 13 is a hinge 1302. The hinge 1302 in the example systems is constructed of the same material (e.g., polymeric material, such as plastic) that makes up the body 1202 of the fiducial 1200. Movement enabled by the hinge 1302 is created by elastic deformation of the underlying material. Moreover, the cross-sectional view of FIG. 13 shows the example hinge 1302 disposed adjacent to the front surface 1204. Extending or projecting toward the back surface 1300 is an example cantilever 1304, and on a distal end of the cantilever 1304 is an annular latch surface 1306.

Still referring to FIG. 13. FIG. 13 also shows an example viewing angle for the fiducial 1200. In particular, the viewing angle for the fiducial 1200 is bounded by a conic frustum 1308 (shown in cross-section) having the smaller diameter equal or about equal to the diameter of the front reflector 1208, and the conic frustum 1308 extending outward along a central axis 1206 normal to the reflective face of front reflector 1208. The side wall of the conic frustum 1308 defines the example limit of the viewing angle, and forms an angle of 45 angular degrees or more (i.e., angle of view 1350) relative to the central axis 1206. In the example case show, the most distal portion of a latch member (distal relative to the latch member's hinge) does not cross into the internal volume of the example conic frustum 1308. Stated otherwise, the most distal portion of a latch member is designed and constructed to not block the view of the front reflector 1208 taking into account the viewing angle for the front reflector 1208.

The example fiducial 1200 further comprises a front latch indicator disposed within the annular channel defined by the front annular latch system (e.g., the latch members 1210, 1212, 1214, and 1216), and likewise defines a back latch indicator disposed within the annular channel defined by the back annular latch system (e.g., the latch members 1224, 1226, 1228, and 1230). In the example system, the front latch indicator 1310 may be a color (e.g., green) painted or applied to the inside surface of the hinges of the latch members. Similarly, the back latch indicator 1312 may be a color (e.g., green) painted or applied to the inside surface of the hinges of the latch members. When the fiducial 1200 is property seated within a reflector receptacle, the front latch indicator 1310 and the back latch indicator 1312 may be visible within the annular channels created by the respective latch members.

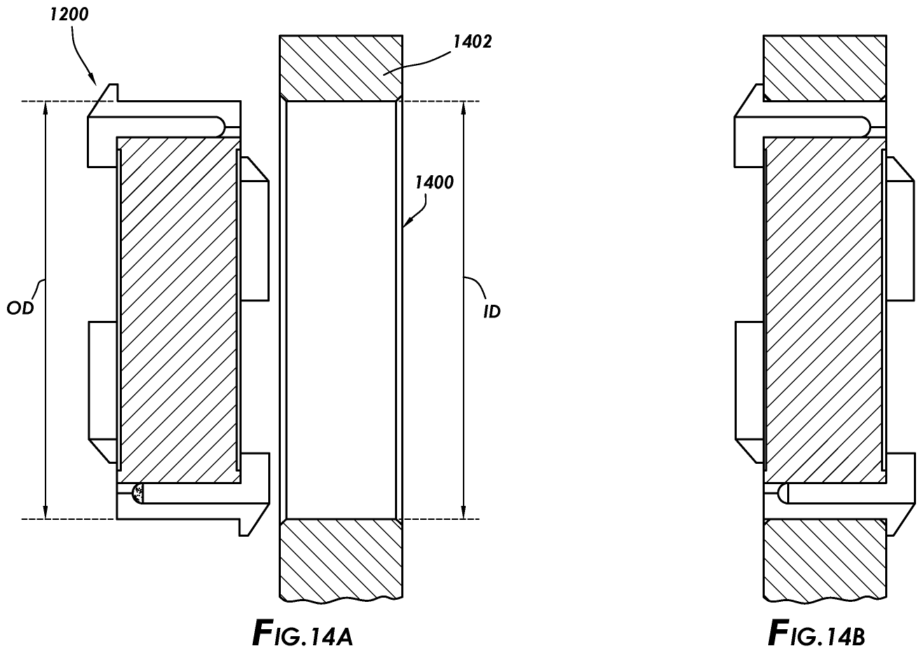

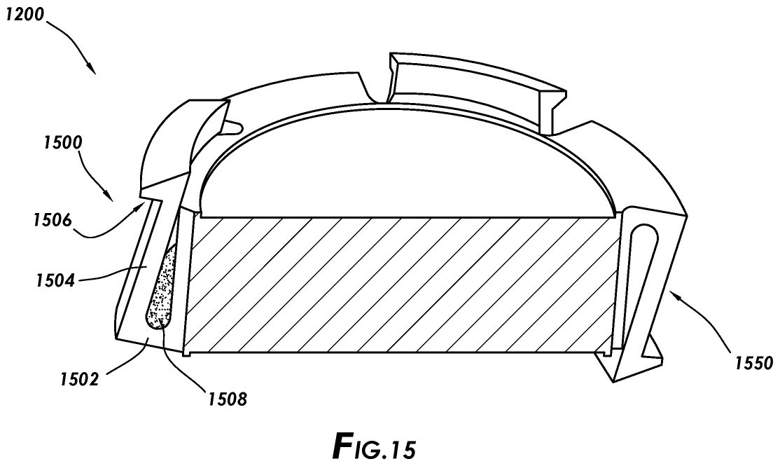

FIG. 14A shows a cross-sectional view of an example fiducial 1200 and reflector receptacle 1400 prior to installation of the fiducial 1200. FIG. 14B shows a cross-sectional view of the fiducial 1200 installed within the reflector receptacle 1400. In particular, FIG. 14A shows a portion of an array structure 1402 defining the example reflector receptacle 1400. The array structure 1402 may be any of the previously discussed array structures, and the reflector receptacle 1400 may be any of the previously discussed reflector receptacles. The example reflector receptacle 1400 defines a through bore with an internal diameter ID. The example fiducial 1200 includes latch members for both the annular latch system associated with the front surface and the annular latch system associated with the back surface. In example cases, the latch members, in a non-deflected state, define an outside diameter OD designed and constructed to form a slip fit with the inside diameter of the reflector receptacle 1400. The example fiducial 1200 may be installed from either side of the array structure 1402. During installation, the fiducial 1200 is pressed into the reflector receptacle 1400. By pressing the fiducial 1200 into the reflector receptacle 1400, the latch members associated with the reflective surface moving through the reflector receptacle are deflected inward, enabling the fiducial to telescope within the reflector receptacle 1400.

Once the latch members clear the opposite face of the reflector receptacle the latch members spring back to have their respective annular latch surfaces abut the outer faces of the reflector receptacle as shown in FIG. 14B. Thus, annular latch surfaces of latch members associated with the front face abut the outer face of the reflector receptacle on that side. Correspondingly, annular latch surfaces of latch members associated with the back face abut the outer face of the reflector receptacle on that side. As between the two sets of latch faces, the fiducial 1400 is held in place within the reflector receptacle. In the situation where only one surface of the fiducial 1200 participates (e.g., 1024, 1022, 1008, 1010, 1012, 1014), one of the reflectors may be eliminated. Alternatively, both reflectors may be incorporated but a mask is incorporated into the array where the reflector is not needed.

The example reflector receptacles shown and discussed to this point implement through bores whose inside surface defines a right-circular cylinder. However, the through bore of the receptacles need not have circular cross-sections. In other examples, the through bores may have oblong cross-sections, or may define cross-sections that define a polygon, such as a square, hexagon, or an octagon. Relatedly, the fiducials shown and discussed to this point implement circular shapes complementary to the example reflector receptacles. However, the fiducials may take any shape complementary to the through bore of the receptacles, such as oblong or a polygon, such as a square, hexagon, or an octagon.

FIG. 15 shows a cross-sectional view of the fiducial 1200 with latch members deflected. During installation of the fiducial 1200 into a reflector receptacle, certain of the latch members are deflected inward toward the central axis 1206 (FIG. 12) as the fiducial 1200 is pressed through the reflector receptacle. In FIG. 15, an example latch member 1500 is shown in a deflected state, and also shows an example latch member 1550 in a deflected state, which may occur when the fiducial 1200 is cocked within the reflector receptacle (e.g., the central axis 1206 of the fiducial 1200 forms a non-zero angle with the longitudinal central axis of the through bore forming the reflector receptacle). Regardless of the reason for having latch members associated with opposite surfaces both being deflected inward, when a latch member is deflected inward the latch indicator for that latch member will be obscured by the cantilever.

The example latch member 1500 defines a hinge 1502, a cantilever 1504, an annular latch surface 1506, and a latch indicator 1508. The cantilever 1504 is deflect inward such that in inside surface of the cantilever abuts the body of the fiducial 1500. When the cantilever 1504 is deflected inward, such obscures the ability of the person installing the fiducial 1200 to see the latch indicator 1508. When the latch indicator 1508 is not visible, then such is an indication that the fiducial 1200 is not fully and/or properly installed. Oppositely, when the fiducial 1200 is held within a reflector receptacle and the latch indicator 1508 is visible, then such is an indication that the fiducial 1200 is fully and/or properly installed.

Returning to FIG. 10. The various embodiments discussed to this point have assumed the example array structures (e.g., array structure 252 or array structure 1102 (FIG. 11)) to define apertures in which are placed the example fiducials discussed with respect the FIGS. 13-13, 14A-B, and 15. However, in the case of fiducial arrays that are single-use items, the fiducial arrays may include fiducials that are integral with the array structure. For example, if the array structure 252 of FIG. 10 is a cast or injection molded component (e.g., injection molded plastic), the reflective surface used to form the fiducial may be cast in place during the injection molding process. In such cases, the reflector receptacles may be the structures around and/or supporting the each reflective surface. In other cases, the reflective surface may be in the form of an adhesive-backed tape or film that is adhered to the location of a reflector receptacle after the array structure is formed. In the case of the reflective surfaces in the form of tape or film adhered to the array structure, the reflector receptacles may be the surfaces to which reflective surface is adhered.

The above discussion is meant to be illustrative of the principles and various embodiments of the present invention. Numerous variations and modifications will become apparent to those skilled in the art once the above disclosure is fully appreciated. For example, in the various embodiments discussed above the elongate outer tube 138 is straight; however, in other examples the elongate outer tube 138 may be curved to provide better access to the underlying tissues. In particular, the elongate outer tube 138 may define a radius of curvature with a tube center of curvature outside the elongate outer tube. In such curved-tube cases, the internal tube or shaft coupled to the cutter is selected to be flexible to enable transferring of the torque from the inner hub 216 to the cutter 134. Moreover, in such curved-tube cases the sleeve 220 of the sleeve connector 202 likewise has a matching radius of curvature to enable the sleeve 220 to telescope over the elongate outer tube 138. In particular, the sleeve 220 may define a radius of curvature with a sleeve center of curvature outside the sleeve 220. Inasmuch as that, when assembled the sleeve 220 is concentrically arranged around the elongate outer tube 138, the tube center of curvature and the sleeve center of curvature would be co-located. In such curved-tube cases, the sleeve connector 202 would couple to the elongate outer tube 138 in only one rotational orientation with respect to the stationary outer hub 214, but otherwise the principles are the same. It is intended that the following claims be interpreted to embrace all such variations and modifications.

1. A resection instrument system comprising:
a handpiece;
a mechanical resection device comprising a stationary outer hub, an elongate outer tube coupled to and extending away from the stationary outer hub, and a cutter disposed at a distal end of the elongate outer tube, the stationary outer hub coupled to the handpiece;
a fiducial array;
a sleeve connector comprising:
a sleeve defining a distal end, a proximal end, and a through bore, the sleeve concentrically arranged with the elongate outer tube;
an array connector coupled to the proximal end of the sleeve, the array connector coupled to the fiducial array.

2. The resection instrument system of claim 1 wherein the array connector further comprises:
a first abutment surface configured to abut a first complementary surface of the fiducial array;
a second abutment surface configured to abut a second complementary surface of the fiducial array; and
a third abutment surface configured to about a third complementary surface of the fiducial array; and
a latch configured to hold the fiducial array in an abutting relationship with the first, second, and third abutment surfaces.

3. The resection instrument system of claim 1 wherein the array connector further comprises:
a slot formed by the array connector; and
a fiducial connector of the fiducial array disposed within the slot.

4. The resection instrument system of claim 1 wherein the array connector further comprises:
a first arm;
a second arm; and
an intersection defined by the first and second arms, and wherein the fiducial connector abuts the intersection.

5. (canceled)

6. The resection instrument system of claim 4 wherein the first arm and the second arm are coplanar, and a plane defined by the first and second arms is parallel to a longitudinal central axis of the through bore of the sleeve.

7. The resection instrument system of claim 1 wherein the array connector further comprises:
a first notch, and a first portion of the fiducial array disposed with the first notch;
a second notch, and a second portion of the fiducial array disposed with the second notch.

8. The resection instrument system of claim 7 wherein the first portion of the fiducial array elastically deforms the first notch and the second portion of the fiducial array elastically deforms the second notch.

9. The resection instrument system of claim 1 wherein the sleeve connector further comprises:
a first latch disposed proximate to a receptacle on the proximal end of the sleeve, the first latch configured to hold the sleeve at a fixed axial position relative to the stationary outer hub when the sleeve is located concentrically around the elongate outer tube; and
a second latch disposed proximate to the receptacle, the second latch configured to hold the sleeve at the fixed axial position relative to the stationary outer hub when the sleeve is concentrically disposed around the elongate outer tube.

10. The resection instrument system of claim 9 further comprising:
the first latch further comprises a first latch arm having a first length measured from a hinge of the first latch; and
the second latch further comprises a second latch arm having a second length measured from a hinge of the second latch, the second length longer than the first length.

11. The resection instrument system of claim 1 wherein the sleeve connector further comprises a counter bore on proximal end of the sleeve, the counter bore defining an inside diameter greater than an outside diameter of the stationary outer hub.

12. The resection instrument of claim 1 further comprising:
the sleeve defines a radius of curvature with a sleeve center of curvature outside the sleeve;
the elongate outer tube a radius of curvature with a tube center curvature outside the elongate outer tube; and
the sleeve center of curvature and the tube center of curvature are co-located.

13. The resection instrument system of claim 1 wherein the fiducial array further comprises:
a first set of reflector receptacles, the first set of reflector receptacles define and reside in a first plane;
a first set of fiducials disposed one each in association with each of the first set of reflector receptacles;
a second set of reflector receptacles, the second set of reflector receptacles define and reside in a second plane;
a second set of fiducials disposed one each in association with each of the second set of reflector receptacles;
a third set of reflector receptacles, the third set of reflector receptacles define and reside in a third plane;
a third set of fiducials disposed one each in association with each of the third set of reflector receptacles; and
an array structure that holds the first set of reflector receptacles, the second set of reflector receptacles, and the third set of reflector receptacles in a fixed orientation.

14. The resection instrument system of claim 13 further comprising at least three reflector receptacles in each of the first, second, and third sets of reflector receptacles.

15. (canceled)

16. The resection instrument system of claim 13 wherein at least one reflector receptacle of the second set of reflector receptacles is shared with the third set of reflector receptacles.

17. The resection instrument system of claim 13 further comprising:
the first set of reflector receptacles defines a first pattern;
the second set of reflector receptacles defines a second pattern different than the first pattern; and
the third set of reflector receptacles defines a third pattern different than the first and second patterns.

18. The resection instrument system of claim 13 wherein the fiducial array further comprises the first plane intersects the second plane, the second plane intersects the third plane, and the third plane intersects the first plane.

19.-20. (canceled)

21. The resection instrument system of claim 1 wherein the fiducial array further comprises a fiducial reflector comprising:
a body defining a front surface, a back surface, and a central axis;
a front reflector disposed on the front surface; and
a first annular latch partially circumscribing the body, the first annular latch comprising a hinge proximate to back surface, a cantilever projecting toward the front surface, and a catch surface parallel to the front surface.

22. The resection instrument system of claim 21 further comprising a second annular latch partially circumscribing the body, the second annular latch comprising a hinge proximate to front back surface, a cantilever projecting toward the back surface, and a catch surface parallel to the back surface.

23.-24. (canceled)

25. The resection instrument system of claim 22:
the first annular latch further comprises a first latch member and a second latch member, each of the first and second latch members comprising a hinge proximate to back surface, a cantilever projecting toward the front surface, and a catch surface parallel to the front surface;
a second annular latch further comprises a third latch member and a fourth latch member, each of the third and fourth latch members comprising a hinge proximate to the front back surface, a cantilever projecting toward the back surface, and a catch surface parallel to the back surface; and
the second latch member disposed between the first and second latch members.

26. The resection instrument system of claim 21 further comprising a latch indicator disposed within an annular channel defined between the first annular latch and the body, the latch indicator visible when the first annular latch is in a non-deflected orientation

27.-49. (canceled)
